(19) United States
(12) Patent Application Publication
(10) Pub. No.: US 2025/0381415 A1
(43) Pub. Date: Dec. 18, 2025

(54) MULTI-PORT CONNECTOR CABLE PANEL

(71) Applicant: LUMEDA INC., Rocky Hill, CT (US)

(72) Inventors: Bryan Outlaw, Broad Brook, CT (US); Trevor MacDougall, South Dartmouth, MA (US); Francesco BIRRITTA, Wethersfield, CT (US); Scott NELSON, Vernon, CT (US); Krenar TASIMI, Fairfield, CT (US); Maxwell GERHARDSON, Agawam, MA (US)

(73) Assignee: Lumeda inc, Rocky Hill, CT (US)

(21) Appl. No.: 18/041,912

(22) PCT Filed: Nov. 6, 2022
(86) PCT No.: PCT/US2022/079365

§ 371 Date: Feb. 16, 2023

(51) Int. Cl. A61N 5/06; A61N 5/067; G02B 6/38; G02B 6/42; G02B 6/44

(52) U.S. Cl. CPC A61N 5/062; A61N 5/067; G02B 6/3887; G02B 6/3893; G02B 6/425; G02B 6/426; G02B 6/44785; A61N 2005/0628; A61N 2005/063

(57) ABSTRACT

In some implementations, a PDT delivery device may include a housing having a front panel, a plurality of therapy light sources positioned within the housing and a multi-port connector panel positioned on a front portion of the housing. The multi-port connector panel having a lid pivotably mounted to a portion of a top wall to selectively close the interior, a plurality of optical fiber connectors positioned in a plurality of fiber connector apertures formed in a back wall coupled to a respective one of the plurality of therapy light sources and coupled to a plurality of optical fibers, a cable support tray to receive a cable containing the plurality of optical fibers, and at least one cable clamp to cooperate with the cable support tray to releasably capture the cable therebetween.

### BACKGROUND

### Field of the Disclosure

The present disclosure relates to an optical multi-port cable connector panel.

### Description of the Related Art

Light therapy can be used for the treatment of conditions in multiple ways. For example, light therapies involve the delivery of a therapeutic light through a fiber optic device placed proximal to or within a target tumor. In many prior art treatment systems the therapeutic light is provided by a laser. It is important to take precautions and use safeguards to protect the users (typically medical professions) from the dangers imposed by lasers.

Light therapies can be combined with prior administration of light sensitizing medication (i.e., photosensitizer) that absorbs the therapeutic light and interacts with surrounding tissue constituents (e.g., oxygen) to generate reactive species that can destroy the target tissue. This form of therapy is known as photodynamic therapy (“PDT”).

What is needed is a PDT system and methods that overcome the shortcomings of the prior art and provide safe and effective use by medical professionals.

### SUMMARY

A system of one or more computers or microprocessors can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions.

In one general aspect, a multi-port connector panel may include a base having opposing side walls connected to a top wall and a bottom wall and a back wall and a neck portion projecting from the bottom wall to define an interior. The multi-port connector panel may also include a lid pivotably mounted to a portion of the top wall to selectively close the interior. The multi-port connector panel may furthermore include a plurality of optical fiber connectors positioned in a plurality of fiber connector apertures formed in the back wall and configured to be selectively coupled to a plurality of optical fibers. The multi-port connector panel may in addition include a cable support tray positioned in a front portion of the neck portion and configured to receive a cable containing the plurality of optical fibers. The multi-port connector panel may moreover include at least one cable clamp positioned in a front portion of the lid and configured to cooperate with the cable support tray to releasably capture the cable therebetween. Other embodiments of this aspect include corresponding computer systems, apparatus, and computer programs recorded on one or more computer storage devices, each configured to perform the actions of the methods.

Implementations may include one or more of the following features. The multi-port connector panel may include a plurality of photodetectors positioned in a plurality of photodetector apertures formed in the back wall and configured to be selectively coupled to a plurality of detector optical fibers positioned in the cable. The multi-port connector panel may include a hinge attached to the top wall and a top portion of the lid and where the lid is configured to pivot about the hinge relative to the base to selectively expose the interior in an open position and to selectively close the interior in a closed position. The multi-port connector panel may include at least one fastener positioned in the front portion of the lid to releasably lock the lid in the closed position. The multi-port connector panel may include at least one interlock switch configured to provide an indication of the open position or the closed position. The multi-port connector panel where the at least one interlock switch may include a lid position switch mounted to the neck portion and a cable position switch positioned within the cable support tray. The multi-port connector panel where the lid position switch is configured to be activated when the lid is in the closed position and where the cable position switch is configured to be activated when the cable is positioned within the cable support tray. The multi-port connector panel where the plurality of optical fiber connectors are each configured to be coupled to a respective one of a plurality of laser light sources and are further configured to couple a respective PDT light from the plurality of laser light sources to the plurality of optical fibers and where the plurality of photodetectors are each configured to produce an electrical signal based on a detected light transmitted from a respective one of the plurality of optical fibers. The multi-port connector panel where the base is configured to mount to a The PDT instrument and where the plurality of laser light sources are housed in the PDT instrument and the plurality of optical fibers and the plurality of optical fibers are configured to be optically coupled to a conformable light applicator. The multi-port connector panel where the plurality of laser light sources are configured to be energized when the lid position switch is activated and when the cable position switch is activated. The multi-port connector panel may include indicia positioned proximate the plurality of optical fiber connectors and the plurality of photodetectors in the base and matching indicia positioned on each of the plurality of optical fibers and the plurality of detector optical fibers. The multi-port connector panel further may include a pair of threaded holes positioned in the front portion of the neck portion and where the at least one faster may include a pair of threaded thumb screws rotatably captured in the front portion of the lid and where the pair of threaded thumb screws cooperate with the pair of threaded holes to releasably lock the lid in the closed position. The multi-port connector panel where the base, the lid and the cable cooperate to form a secure light enclosure when the at least one fastener releasably locks the lid in the closed position. The multi-port connector panel where the plurality of optical fibers and the plurality of detector optical fibers include an excess fiber length between an exit end of the cable and the plurality of optical fiber connectors and the plurality of photodetectors and where the at least one cable clamp and the cable support tray releasably capture the cable and provide strain relief to the plurality of optical fibers and the plurality of detector optical fibers in the closed position. The multi-port connector panel where the hinge may include: a pair of base leaves positioned on a top portion of the base, the pair of base leaves including a respective tang; a pair of lid leaves positioned on a top portion of the lid, the pair of lid leaves including a respective detent; and where the respective tangs are configured to cooperate with the respective detent to releasable lock the lid in the open position. The multi-port connector panel may include a photodetector clamp to securely position the plurality of optical fiber connectors in the plurality of fiber connector apertures and the plurality of photodetectors in the plurality of photodetector apertures. The multi-port connector panel where the cable support tray includes a locking mechanism configured releasably capture the cable in the cable support tray. The multi-port connector panel where the locking mechanism may include a snap fit. The multi-port connector panel where the snap fit may include a right shoulder and a left shoulder positioned on a top portion of the cable support tray and where the cable has an outer cable diameter and where a distance between the right shoulder and the left shoulder is smaller than the outer cable diameter configured to provide an interference fit between the right shoulder and the left shoulder and the cable. Implementations of the described techniques may include hardware, a method or process, or a computer tangible medium.

In one general aspect, a PDT instrument may include a housing having a front panel. The PDT instrument may also include a plurality of therapy light sources positioned within the housing. Instrument may furthermore include a multi-port connector panel positioned on a front portion of the housing having: a base having opposing side walls connected to a top wall and a bottom wall and a back wall and a neck portion projecting from the bottom wall to define an interior; a lid pivotably mounted to a portion of the top wall to selectively close the interior; a plurality of optical fiber connectors positioned in a plurality of fiber connector apertures formed in the back wall coupled to a respective one of the plurality of therapy light sources and configured to be selectively coupled to a plurality of optical fibers; a cable support tray positioned in a front portion of the neck portion and configured to receive a cable containing the plurality of optical fibers; and at least one cable clamp positioned in a front portion of the lid and configured to cooperate with the cable support tray to releasably capture the cable therebetween. Other embodiments of this aspect include corresponding computer systems, apparatus, and computer programs recorded on one or more computer storage devices, each configured to perform the actions of the methods.

Implementations may include one or more of the following features. The PDT instrument may include a plurality of photodetectors positioned in a plurality of photodetector apertures formed in the back wall and configured to be selectively coupled to a plurality of detector optical fibers positioned in the cable. The PDT instrument may include a hinge attached to the top wall and a top portion of the lid and where the lid is configured to pivot about the hinge relative to the base to selectively expose the interior in an open position and to selectively close the interior in a closed position. The PDT instrument may include at least one fastener positioned in the front portion of the lid to releasably lock the lid in the closed position. The PDT instrument may include at least one interlock switch configured to provide an indication of the open position or the closed position. The PDT instrument where the at least one interlock switch may include a lid position switch mounted to the neck portion and a cable position switch positioned within the cable support tray. The PDT instrument where the lid position switch is configured to be activated when the lid is in the closed position and where the cable position switch is configured to be activated when the cable is positioned within the cable support tray. The PDT instrument where the plurality of photodetectors are each configured to produce an electrical signal based on a detected light transmitted from a respective one of the plurality of optical fibers. The PDT instrument where the plurality of optical fibers are configured to be optically coupled to a conformable light applicator. The PDT instrument where the plurality of therapy light sources are configured to be energized when the lid position switch is activated and when the cable position switch is activated. The PDT instrument may include indicia positioned proximate the plurality of optical fiber connectors and the plurality of photodetectors in the base and matching indicia positioned on each of the plurality of optical fibers and the plurality of detector optical fibers. The PDT instrument may include a key switch mounted in the front portion configured to power on the PDT instrument. The PDT instrument may include a laser power indicator light positioned on the front portion of the housing and configured to illuminate when the key switch is in an on positioned, the lid position switch is activated and the cable position switch is activated. The PDT instrument may include an emergency stop button positioned on the front portion of the housing and configured to shut down the PDT instrument when activated. The PDT instrument further may include a pair of threaded holes positioned in the front portion of the neck portion and where the at least one faster may include a pair of threaded thumb screws rotatably captured in the front portion of the lid and where the pair of threaded thumb screws cooperate with the pair of threaded holes to releasably lock the lid in the closed position. The PDT instrument where the base, the lid and the cable cooperate to form a secure light enclosure when the at least one fastener positioned releasably locks the lid in the closed position. The PDT instrument where the plurality of optical fibers and the plurality of detector optical fibers include an excess fiber length between an exit end of the cable and the plurality of optical fiber connectors and the plurality of photodetectors and where the at least one cable clamp and the cable support tray releasably capture the cable and provide strain relief to the plurality of optical fibers and the plurality of detector optical fibers in the closed position. The PDT instrument where the hinge may include: a pair of base leaves positioned on a top portion of the base, the pair of base leaves including a respective tang; a pair of lid leaves positioned on a top portion of the lid, the pair of lid leaves including a respective detent; and where the respective tangs are configured to cooperate with the respective detent to releasable lock the lid in the open position. The PDT instrument may include a photodetector clamp to securely position the plurality of optical fiber connectors in the plurality of fiber connector apertures and the plurality of photodetectors in the plurality of photodetector apertures. The PDT instrument may include: a microprocessor positioned within the housing configured to execute a treatment plan; and a monitor screen positioned on the front portion of the housing. The PDT instrument where the monitor screen may include a graphical user interface configured to enable an user to control a plurality of parameters related to the treatment plan and at least on function of the plurality of therapy light sources. The PDT instrument may include a data port positioned on the housing configured to provide communication between the microprocessor and an external device. The PDT instrument where the external device is a data storage device configured to store the treatment plan. The PDT instrument where the PDT instrument is configured to deliver the treatment plan to a patient via a conformable light applicator. The PDT instrument where the cable support tray includes a locking mechanism configured releasably capture the cable in the cable support tray. The PDT instrument where the locking mechanism may include a snap fit. The PDT instrument where the snap fit may include a right shoulder and a left shoulder positioned on a top portion of the cable support tray and where the cable has an outer cable diameter and where a distance between the right shoulder and the left shoulder is smaller than the outer cable diameter configured to provide an interference fit between the right shoulder and the left shoulder and the cable. Implementations of the described techniques may include hardware, a method or process, or a computer tangible medium.

In one general aspect, a method may include providing a PDT instrument having a housing having a front panel, a plurality of therapy light sources positioned within the housing, a multi-port connector panel positioned on a front portion of the housing having a base having opposing side walls connected to a top wall and a bottom wall and a back wall and a neck portion projecting from the bottom wall to define an interior, a lid pivotably mounted to a portion of the top wall to selectively close the interior, a plurality of optical fiber connectors positioned in a plurality of fiber connector apertures formed in the back wall, a cable support tray positioned in a front portion of the neck portion, and at least one cable clamp positioned in a front portion of the lid. The method may also include selectively coupling each of the plurality of optical fiber connectors to a respective one of the plurality of therapy light source. The method may furthermore include selectively coupling each of the plurality of optical fiber connectors to a respective one of a plurality of optical fibers. Other embodiments of this aspect include corresponding computer systems, apparatus, and computer programs recorded on one or more computer storage devices, each configured to perform the actions of the methods.

Implementations may include one or more of the following features. The method where the PDT instrument further may include a plurality of photodetectors positioned in a plurality of photodetector apertures formed in the back wall and where the method further may include selectively coupling each of the plurality of photodetectors to a respective one of a plurality of detector optical fibers positioned in a cable. The method where the PDT instrument may include a lid position switch mounted to the neck portion and a cable position switch positioned within the cable support tray, the method may include positioning the cable within the cable support tray and activating the cable position switch, pivoting the lid into a closed position and activating the lid position switch, clamping the cable between the at least one cable clamp and the cable support tray, and releasably locking the lid in the closed position. The method may include optically coupling the cable to a conformable light applicator. The method where optically coupling the cable to a conformable light applicator may include optically coupling each of the plurality of optical fibers to a respective one of a plurality of light emitters positioned in the conformable light applicator and optically coupling each of the plurality of detector optical fibers to a respective one of a plurality of fiber isotropic detectors positioned in the conformable light applicator. The method may include developing a treatment plan for a target tissue of the patient where the treatment plan includes a total dosage of a therapy light, and inputting the treatment plan into the PDT instrument. The method may include positioning the conformable light applicator proximate the target tissue of the patient. The method may include powering on the PDT instrument, delivering the therapy light to the target tissue of the patient using the plurality of light emitters, monitoring the delivering of the therapy light to the target tissue of the patient using the plurality of fiber isotropic detectors to determine a dosage of delivered therapy light, and comparing the dosage of delivered therapy light to the total dosage. The method may include completing the treatment plan when the dosage of delivered therapy light is substantially equal to the total dosage. The method may include illuminating a laser power indicator light when the PDT instrument is powered on, the lid position switch is activated and the cable position switch is activated. The method may include controlling the treatment plan using a graphical user interface. The method may include providing strain relief to the plurality of optical fibers and the plurality of detector optical fibers in the closed position. The method may include forming a secure light enclosure in the closed position. Implementations of the described techniques may include hardware, a method or process, or a computer tangible medium.

### BRIEF DESCRIPTION OF THE DRAWINGS

FIG. 1 is a schematic representation of a PDT instrument including a multi-port connector panel in accordance with the present disclosure;

FIG. 2 is a right side isometric view of a PDT instrument including a multi-port connector panel in accordance with the present disclosure;

FIG. 3 is a frontal view of a PDT instrument including a multi-port connector panel in accordance with the present disclosure;

FIG. 4 is a right side isometric view of a multi-port connector panel in accordance with the present disclosure;

FIG. 5 is a right side isometric view of a PDT instrument including a multi-port connector panel in accordance with the present disclosure;

FIG. 6 is a right side isometric view of a PDT instrument including a multi-port connector panel in accordance with the present disclosure;

FIG. 7 is a right side isometric view of a multi-port connector panel in accordance with the present disclosure; and

FIG. 8 is a frontal view in partial section of a multi-port connector panel in accordance with the present disclosure.

### DETAILED DESCRIPTION

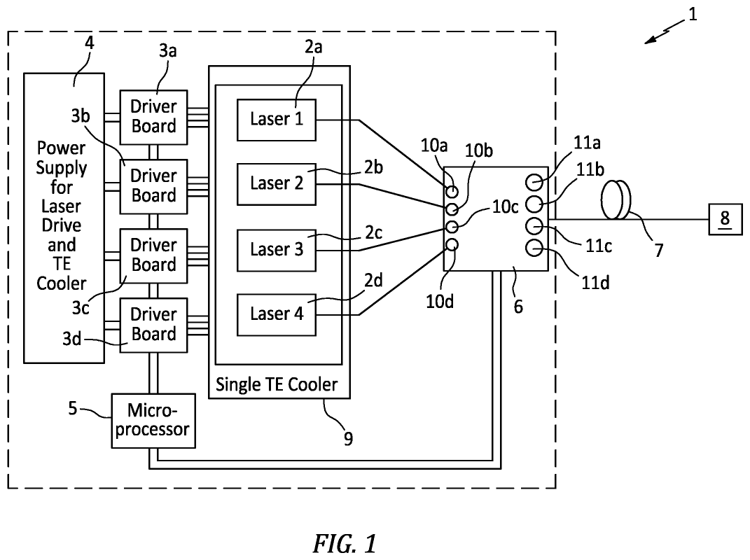

Now referring to FIG. 1, there is shown a PDT instrument 1 comprising a plurality of therapy light sources 2a-2d, a plurality of driver boards 3a-3d, a power supply 4, a microprocessor 5, a multi-port connector assembly 6, a multi-channel cable 7 and a light conformable light applicator 8. Therapy light sources 2a-2d can comprise a plurality of laser light sources as disclosed herein after and are electrically coupled to respective driver boards 3a-3d and to power supply 4. The 4-laser array of therapy light sources 2a-2d can comprise laser model number UP635F15FN-3.0W commercially available from CAS Laser, China and which can produce a continuous wave of constant power output of 2 Watts per laser at 630 nm +/−3 nm. PDT instrument 1 further includes microprocessor 5 wherein the microprocessor can be comprised of an RS232 interface and a computer processer capable of storing executable programs and data and running software to perform the functions disclosed herein. PDT instrument 1 optionally includes cooling device 9 electrically coupled to power supply 4 to maintain therapy light sources 2a-2d at a predetermined operating temperature. The executable programs and data can be stored in any known type of memory device. Cooling device 9 can comprise a thermoelectric cooler and a heat sink or other suitable cooling device capable of maintaining therapy light sources 2a-2d at a predetermined operating temperature. Multi-port connector assembly 6 includes fiber connectors 10a-10d and photodetectors 11a-11d as well as other features as will be disclosed in more detail herein after. Multi-channel cable 7 includes a plurality of delivery optical fibers respectively coupled to fiber connectors 10a-10d and, at an exit end of the delivery optical fibers, to conformable light applicator 8 as well as a plurality of detector optical fibers respectively optically coupled to photodetectors 11a-11d and the optical conformable light applicator. Therapy light sources 2a-2d are respectively optically coupled to fiber connectors 10a-10d and photodetectors 11a-11d are electrically connected to microprocessor 5. In operation, a therapy plan can be determined for a target area of patient that includes a desired irradiance pattern and dosimetry for the pattern. The therapy plan can be entered into processor 5 where algorithms can determine optimum control of the therapy light sources 2a-2d to produce a desired irradiance pattern and dosimetry from light conformable light applicator 8. The light conformable light applicator 8 can be positioned against (or within) the target area of patient and oriented using methods disclosed herein or other suitable methods. The therapy plan is started by driver boards 3a-3d sending a respective start condition control signal to therapy light sources 2a-2d intended to produce the desired irradiance pattern and dosimetry. Light conformable light applicator 8 provides a monitoring light signal of detected light to the respective photodetectors 11a-11d in real time via detector optical fibers of multi-channel cable 7. Photodetectors 11a-11d provide a respective monitoring signal to processor 5 wherein the processor compares the start condition control signals to the monitoring signals and sends a respective updated control signal to driver boards 3a-3d. It is important to note that not all of the driver boards 3a-3d and therapy light sources 2a-2d may be necessary at any point in time during the operation of PDT instrument 1. The operation of monitoring, comparing and delivering of therapy light is continued until the therapy plan is completed within acceptable limits. Such acceptable limits can be pre-programmed into processor 5 or can be input into the processor by a user. It should be appreciated by those skilled in the art that cooling device 9 limits drift that can occur when lasers are operated for extended periods and can therefore remove one of the variables that can cause PDT instrument 1 from producing a desired irradiance pattern and dosimetry in accordance with the therapy plan.

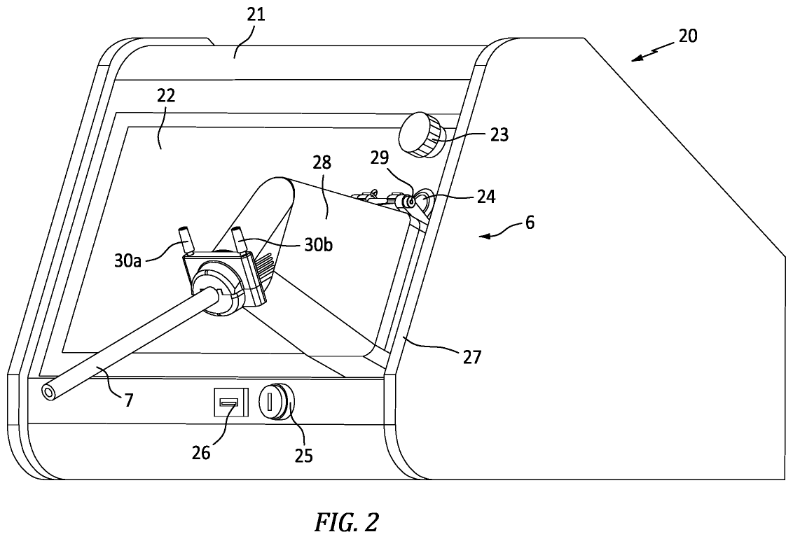

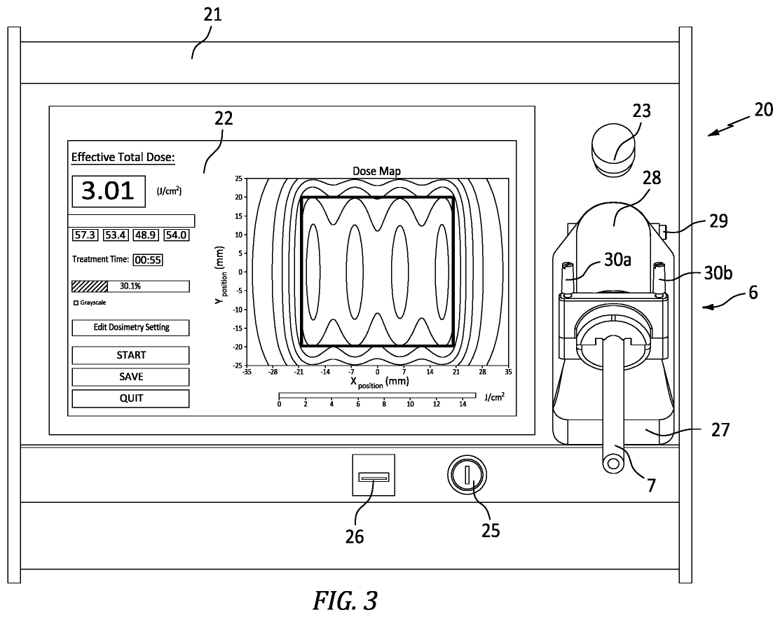

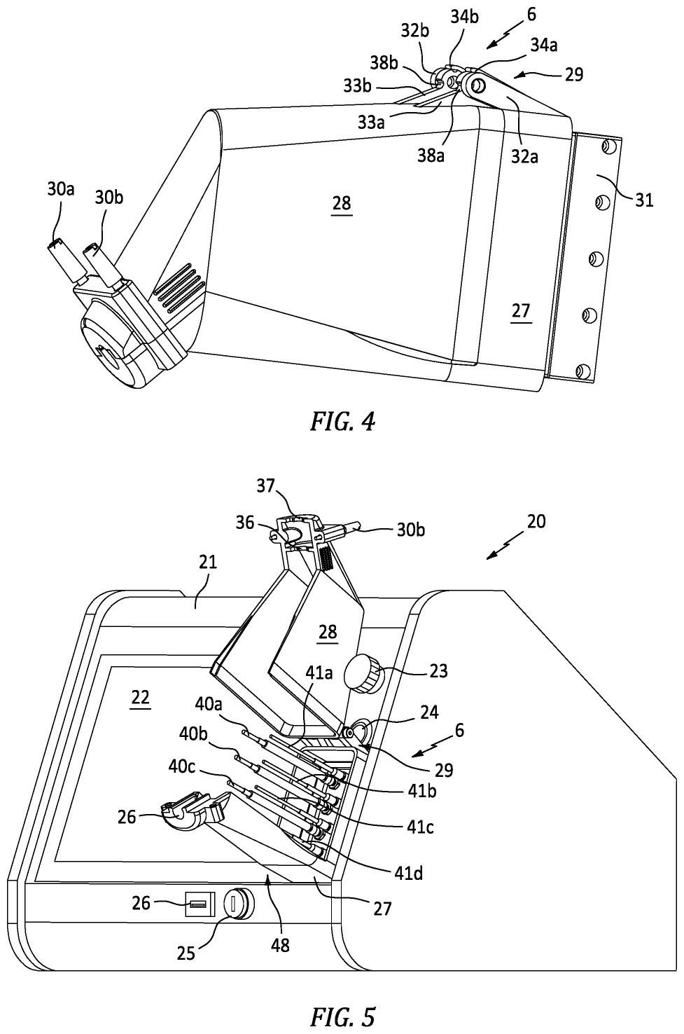

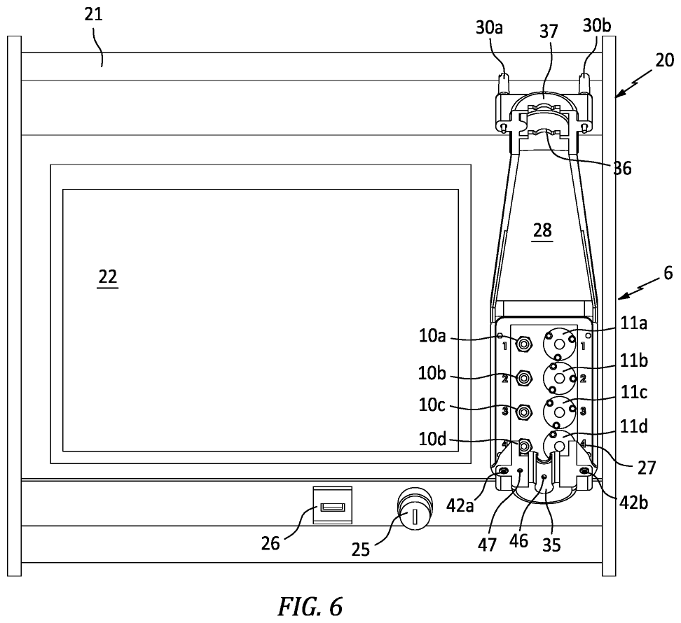

Referring to FIG. 2, there is shown an isometric view of an implementation of PDT instrument 1 (FIG. 1) in the form of PDT Instrument 20. As part of the same implementation, reference can be made to FIG. 3 showing a front view of PDT instrument 20 and FIG. 4 showing an isometric view of multi-port connector assembly 6 mounted within an aperture (not shown) on a front portion of PDT instrument 20. It should be noted that with reference to various figures referenced herein, similar elements will have consistent numbering and nomenclature for purposes of brevity and clarity. With reference to FIGS. 2-4, PDT instrument includes housing 21, monitor screen 22, emergency stop button 23, laser power indicator 24, key switch 25, connector port 26 (which can comprise a USBI connector port), and multi-port connector assembly 6. Also shown in the figures is a portion of multi-channel cable 7. Multi-port connector assembly 6 is fixedly attached to a front panel of housing 21 via base 27 and includes hinged lid 28 pivotally attached to the base by hinge 29. As will be disclosed in more detail herein after, hinged lid 28 pivots between a closed position (FIGS. 2-4) and an open position (FIGS. 5-7) to secure multi-channel cable 7 in the closed position and to allow access to the interior of multi-port connector assembly 6 in the open position. Multi-port connector assembly 6 further contains at least one fastener in the form of threaded thumb screws forming lid locks 30a, 30b rotatably captured within hinged lid 28 that cooperated with mating threaded holes (42a, 42b in FIGS. 6,7). With specific reference to FIG. 4, multi-port connector assembly 6 further includes photodetector clamp 31 to securely position photodetectors 11a-11d and fiber connectors 10a-10d as will be disclosed in more detail herein after. Hinge 29 is comprised of base leaves 32a, 32b integrated into base 27 and lid leaves 33a, 33b integrated into hinged lid 28 that cooperate to allow the hinged lid to be rotated relative to the base. Base leaves 33a, 33b include respective tangs 34a, 34b that engage with detents 38a, 38b integrated into lid leaves 33a, 33b to form a hold open lock to releasably secure hinged lid 28 in the open position as will be disclosed in more detail. As will be disclosed in more detail herein after, base 27 includes a cavity formed by a top wall, a pair of opposing side walls and a bottom wall wherein neck portion 48 projects from the bottom wall.

Although not shown in FIGS. 2-8, the plurality of therapy light sources 2a-2d, the plurality of driver boards 3a-3d, the power supply 4, the microprocessor 5 and other components, such as power cords, circuit boards, cooling fans and various connectors are positioned within housing 21 of PDT instrument 20. It should be noted that although an implementation is being disclosed having four therapy light sources 2a-2d and four driver boards 3a-3d, the PDT instrument 20 of the present disclosure is not so limited and may comprise any number of such components.

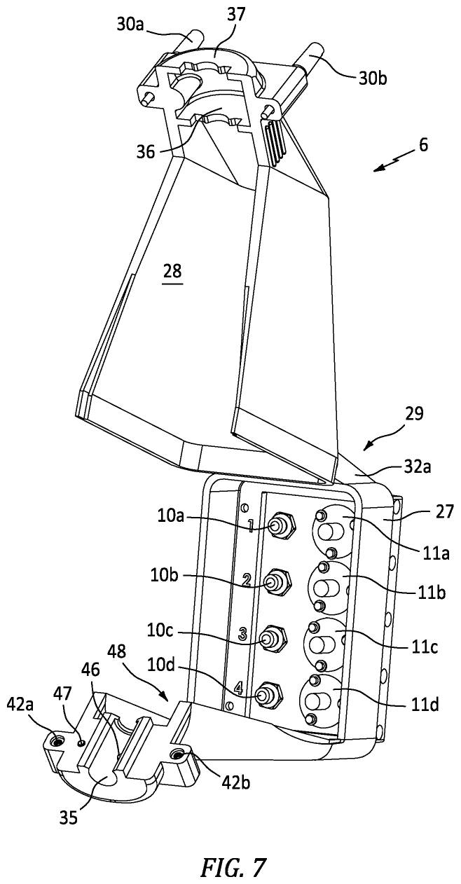

Now with reference to FIGS. 5-7, there is shown PDT instrument 20 with hinged lid 28 of multi-port connector assembly 6 in the open and locked position. With hinged lid 28 in the open position, additional features of multi-port connector assembly 6 are revealed including cable support tray 35 integrated into a front portion of neck portion 48 of base 27 and inner cable clamp 36 and outer cable clamp 37 integrated into hinged lid 28. With specific reference to FIG. 5, in implementations of PDT instrument 20 of the current disclosure, multi-channel cable 7 is comprised of delivery optical fibers 40a-40d (40a-40c shown in section from multi-channel cable 7, 40d not shown) and detector optical fibers 41a-41d (41a-41d shown in section from multi-channel cable 7). For much of the length of multi-channel cable 7, delivery optical fibers 40a-40d and detector optical fibers 41a-41d are contained within an encapsulation layer 70 (FIG. 7) and until they exit the exit end of the encapsulation layer leaving an overstuff amount, and appropriate connectors are optically coupled to the fibers. It should be appreciated by those skilled in the art that cable support tray 35 integrated into base 27 and inner cable clamp 36 and outer cable clamp 37 cooperate to releasably capture multi-channel cable 7 between the support tray and the inner cable clamp and outer cable clamp when hinged lid 28 is in the closed position and during use of PDT instrument 20.

With specific reference to FIGS. 6, 7, multi-channel connector assembly 6 further includes output optical fiber connectors 10a-10d and input photodetectors 11a-11d positioned within respective fiber connector apertures and photodetector apertures in a back wall of base 27. As disclosed herein above with reference to FIG. 1, therapy light sources 2a-2d are coupled to a respective backside (not shown) of output fiber connectors 10a-10d. Output fiber connectors 10a-10d can comprise a commercially available SubMiniature version A (SMA) 905 optical connector and delivery optical fibers 40a-40d can include a mating connector each to releasably optically couple the optical fibers to the therapy light sources via the output fiber connectors. Delivery optical fibers 40a-40d are optically coupled to light emitters (not shown) which can comprise cylindrical light emitters positioned in light conformable light applicator 8 (FIG. 1). Light conformable light applicator 8 can also comprise fiber isotropic detectors, i.e. a IP85 detector commercially available from Medlight, SA optically coupled to detector optical fibers 41a-41d. The therapy light is detected using the isotropic light detectors and transmitted back to PDT instrument 20 via detector optical fibers 41a-41d within multi-channel cable 7 which are optically connected to input photodetectors 11a-11d using SMA 905 connectors. Input photodetectors 11a-11d can each comprise a silicon detector and amplifier, such as and Oz Optics part number PD-05-450/1100-M-PD:5 mm to produce an electrical signal proportional to the therapy light detected. The electrical signals are coupled to microprocessor 5 and processed using an algorithm to derive accurate absolute dose rate (mW/cm2) and total dosage (J/cm2) values of delivered therapy light from light conformable light applicator 8.

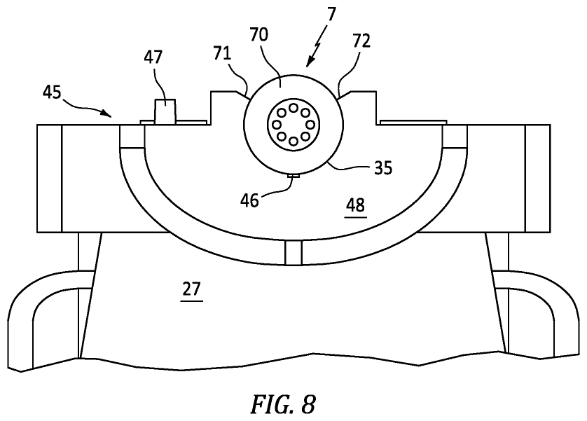

Also shown in FIGS. 6, 7 are mating threaded holes 42a, 42b which, as disclosed herein above, which respectively engage with the thumb screws of lid locks 30a, 30b to releasably secure hinged lid 28 to base 27 in a closed position. Still referring to FIGS. 6, 7 and with additional reference to FIG. 8, there is shown instrument interlock switch system 45 comprised of cable position switch 46 positioned in cable support tray 35 and lid position switch 47 positioned in a neck portion 48 of base 27. As shown in the figure, cable position switch 46 is in the depressed condition (closed) and lid position switch 47 is in the raised position (open). It should be apparent to those skilled in the art that PDT instrument 20 can pose a danger to medical personnel if therapy light sources 2a-2d, which can produce 2W or more of energy, were activated without delivery optical fibers 40a-40d coupled to output fiber connectors 10a-10d including when hinged lid 28 is in the open position. To improve the safety and operability of PDT instrument 20 cable position switch 46 and lid position switch 47 are electrically coupled to microprocessor 5 to prevent the inadvertent powering of therapy light sources 2a-2d. Cable position switch 46 is a normally open contact switch that is closed when cable 7 is positioned within cable support tray 35 with encapsulation layer 70 depressing the cable position switch as shown in FIGS. 7. Similarly lid position switch 47 comprises a normally open contact switch that is closed when hinged lid 28 is threadably secured to base 27 using lid locks 30a, 30b. Micro-processor 5 includes logic that ensures that both cable position switch 46 and lid position switch 47 are in a closed position prior to operation of therapy light sources 2a-2d. In this manner, cable position switch 46 and lid position switch 47 provide an indication of the position of the lid. It should be appreciated by those skilled in the art that hinged lid 28 and base 27 cooperate with cable 7 to provide a secure light enclosure when the cable is clamped within cable support tray 35 and the hinged lid is threadably secured to the base using lid locks 30a, 30b such that no therapy light can escape multi-port connector assembly 6.

While still referring to FIG. 8, it is shown that cable support tray 35 has an arcuate shape that matches the round outside diameter of encapsulation layer 70 of cable 7. The arcuate shape of cable support tray is slightly more than semi circular and left shoulder 71 and right shoulder 72 are separated by a distance to form a snap fit which functions to releasably lock cable 7 within the cable support tray and comprises a locking mechanism. In operation, cable 7 is aligned with cable support tray 35 and is inserted into the cable support tray where the outer cable diameter is comprised of the outer diameter of encapsulation layer 70 contacts left shoulder 71 and right shoulder 72 which comprises a smaller dimension of the cable support tray at its top portion. Cable 7 is then forceably positioned in the bottom of cable support tray 35 with encapsulation layer 70 compressing as it passes through left shoulder 71 and right shoulder 72 of the cable support tray in an interference fit and becoming releasably locked within the cable support tray. Cable 7 can be removed from cable support tray 35 by reversing the operation procedure disclosed immediately herein above.

Monitor screen 22 can comprise a graphical user interface (GUI) to allow medical personnel to control the relevant parameters related to the treatment and functionality of the laser device. PDT instrument 20 can be operated with sequential screens guiding a user to perform required tasks before actual treatment. In operation, a user threadably releases lid locks 30a, 30b and positions hinged lid 28 in the open position with tangs 34a, 34b in the detents of lid leaves 33a, 33b to releasably lock the hinged lid in the open position. The user then connects delivery optical fibers 40a-40d output fiber connectors 10a-10d and detector optical fibers 41a-41d to input photodetectors 11a-11d and positions encapsulation 7 into cable support tray 35 thereby closing cable position switch 46. Indicia can be included on the fibers and connectors to ensure proper connections are made. Any excess fiber length of optical fiber (overstuff) can be positioned in fiber tray 48 (FIG. 7) of base 27. Hinged lid 28 is then pivoted to the closed position and lid locks 30a, 30b are tightened thereby closing lid position switch 47 capturing cable 7 there between and providing strain relief to the fibers. With external power supplied to PDT instrument 20, the user can use key switch 25 to power on the instrument. PDT instrument 20 may require user login and other initial input and then microprocessor 5 can perform diagnostic checks of the instrument to ensure proper operation of the various components. The user, using the GUI on monitor screen (or a keyboard) can input a treatment plan including a target dose (J/cm2) and a dose rate (mW/cm2) and a total dosage of delivered therapy light. The treatment plan can further be uploaded into instrument via an external device using connector port 26 for communication with the microprocessor wherein the external device can comprise a data storage device. Medical personnel can place conformable light applicator in a body of patient against an area of interest for PDT treatment. The user can then, using the GUI, start the treatment plan. The microprocessor will command the driver boards 3a-3d to power the therapy light sources 2a-2d to deliver therapy light to the conformable light applicator 8 and to the patient and laser power indicator light 24 will be illuminated. PDT instrument 20 can display the progress of the treatment plan on the GUI. The user can shut down the PDT instrument 20 using the GUI or the emergency stop button 23 wherein the PDT instrument's internal components will be powered down and laser emission will be disabled. PDT instrument 20 will continue to provide delivered therapy light to the patient until the total dosage is reached. Microprocessor 5 will then power down components of the instrument and can save information pertaining to the actual treatment cycle.

The foregoing disclosure provides illustration and description but is not intended to be exhaustive or to limit the implementations to the precise form disclosed. Modifications may be made in light of the above disclosure or may be acquired from practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code-it being understood that software and hardware can be used to implement the systems and/or methods based on the description herein. As used herein, satisfying a threshold may, depending on the context, refer to a value being greater than the threshold, greater than or equal to the threshold, less than the threshold, less than or equal to the threshold, equal to the threshold, and/or the like, depending on the context. Although particular combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

What is claimed is:

1. A multi-port connector panel comprising:
a base having opposing side walls connected to a top wall and a bottom wall and a back wall and a neck portion projecting from the bottom wall to define an interior;
a lid pivotably mounted to a portion of the top wall to selectively close the interior;
a plurality of optical fiber connectors positioned in a plurality of fiber connector apertures formed in the back wall and configured to be selectively coupled to a plurality of optical fibers;
a cable support tray positioned in a front portion of the neck portion and configured to receive a cable containing the plurality of optical fibers; and
at least one cable clamp positioned in a front portion of the lid and configured to cooperate with the cable support tray to releasably capture the cable therebetween.

2. The multi-port connector panel of claim 1, further comprising a plurality of photodetectors positioned in a plurality of photodetector apertures formed in the back wall and configured to be selectively coupled to a plurality of detector optical fibers positioned in the cable.

3. The multi-port connector panel of claim 2, further comprising a hinge attached to the top wall and a top portion of the lid and wherein the lid is configured to pivot about the hinge relative to the base to selectively expose the interior in an open position and to selectively close the interior in a closed position.

4. The multi-port connector panel of claim 3 further comprising at least one fastener positioned in the front portion of the lid to releasably lock the lid in the closed position.

5. The multi-port connector panel of claim 4 further comprising at least one interlock switch configured to provide an indication of the open position or the closed position.

6. The multi-port connector panel of claim 5 wherein the at least one interlock switch comprises a lid position switch mounted to the neck portion and a cable position switch positioned within the cable support tray.

7. The multi-port connector panel of claim 6 wherein the lid position switch is configured to be activated when the lid is in the closed position and wherein the cable position switch is configured to be activated when the cable is positioned within the cable support tray.

8. The multi-port connector panel of claim 1 wherein the cable support tray includes a locking mechanism configured releasably capture the cable in the cable support tray.

9. The multi-port connector panel of claim 8 wherein the locking mechanism comprises a snap fit.

10. The multi-port connector panel of claim 9 wherein the snap fit comprises a right shoulder and a left shoulder positioned on a top portion of the cable support tray and wherein the cable has an outer cable diameter and wherein a distance between the right shoulder and the left shoulder is smaller than the outer cable diameter configured to provide an interference fit between the right shoulder and the left shoulder and the cable.

11. The multi-port connector panel of claim 4 further comprises a pair of threaded holes positioned in the front portion of the neck portion and wherein the at least one fastener comprises a pair of threaded thumb screws rotatably captured in the front portion of the lid and wherein the pair of threaded thumb screws cooperate with the pair of threaded holes to releasably lock the lid in the closed position.

12. The multi-port connector panel of claim 7 wherein the plurality of optical fiber connectors are each configured to be coupled to a respective one of a plurality of laser light sources and are further configured to couple a respective PDT light from the plurality of laser light sources to the plurality of optical fibers and wherein the plurality of photodetectors are each configured to produce an electrical signal based on a detected light transmitted from a respective one of the plurality of optical fibers.

13. The multi-port connector panel of claim 12 wherein the base is configured to mount to a PDT instrument and wherein the plurality of laser light sources are housed in the PDT instrument and the plurality of optical fibers and the plurality of optical fibers are configured to be optically coupled to a conformable light applicator.

14. The multi-port connector panel of claim 13 wherein the plurality of laser light sources are configured to be energized when the lid position switch is activated and when the cable position switch is activated.

15. The multi-port connector panel of claim 12 further comprising indicia positioned proximate the plurality of optical fiber connectors and the plurality of photodetectors in the base and matching indicia positioned on each of the plurality of optical fibers and the plurality of detector optical fibers.

16. The multi-port connector panel of claim 3 wherein the plurality of optical fibers and the plurality of detector optical fibers include an excess fiber length between an exit end of the cable and the plurality of optical fiber connectors and the plurality of photodetectors and wherein the at least one cable clamp and the cable support tray releasably capture the cable and provide strain relief to the plurality of optical fibers and the plurality of detector optical fibers in the closed position.

17. The multi-port connector panel of claim 3 wherein the hinge comprises:
a pair of base leaves positioned on a top portion of the base, the pair of base leaves including a respective tang;
a pair of lid leaves positioned on a top portion of the lid, the pair of lid leaves including a respective detent; and
wherein the respective tangs are configured to cooperate with the respective detent to releasable lock the lid in the open position.

18. The multi-port connector panel of claim 4 wherein the base, the lid and the cable cooperate to form a secure light enclosure when the at least one fastener releasably locks the lid in the closed position.

19. The multi-port connector panel of claim 2 further comprising a photodetector clamp to securely position the plurality of optical fiber connectors in the plurality of fiber connector apertures and the plurality of photodetectors in the plurality of photodetector apertures.

20. A PDT instrument comprising:
a housing having a front panel;
a plurality of therapy light sources positioned within the housing;
a multi-port connector panel positioned on a front portion of the housing comprising:
a base having opposing side walls connected to a top wall and a bottom wall and a back wall and a neck portion projecting from the bottom wall to define an interior;
a lid pivotably mounted to a portion of the top wall to selectively close the interior;
a plurality of optical fiber connectors positioned in a plurality of fiber connector apertures formed in the back wall coupled to a respective one of the plurality of therapy light sources and configured to be selectively coupled to a plurality of optical fibers;
a cable support tray positioned in a front portion of the neck portion and configured to receive a cable containing the plurality of optical fibers; and
at least one cable clamp positioned in a front portion of the lid and configured to cooperate with the cable support tray to releasably capture the cable therebetween.

21. The PDT instrument of claim 20, further comprising a plurality of photodetectors positioned in a plurality of photodetector apertures formed in the back wall and configured to be selectively coupled to a plurality of detector optical fibers positioned in the cable.

22. The PDT instrument of claim 21, further comprising a hinge attached to the top wall and a top portion of the lid and wherein the lid is configured to pivot about the hinge relative to the base to selectively expose the interior in an open position and to selectively close the interior in a closed position.

23. The PDT instrument of claim 22 further comprising at least one fastener positioned in the front portion of the lid to releasably lock the lid in the closed position.

24. The PDT instrument of claim 23 further comprising at least one interlock switch configured to provide an indication of the open position or the closed position.

25. The PDT instrument of claim 24 wherein the at least one interlock switch comprises a lid position switch mounted to the neck portion and a cable position switch positioned within the cable support tray.

26. The PDT instrument of claim 25 wherein the lid position switch is configured to be activated when the lid is in the closed position and wherein the cable position switch is configured to be activated when the cable is positioned within the cable support tray.

27. The PDT instrument of claim 20 wherein the cable support tray includes a locking mechanism configured releasably capture the cable in the cable support tray.

28. The PDT instrument of claim 27 wherein the locking mechanism comprises a snap fit.

29. The PDT instrument of claim 28 wherein the snap fit comprises a right shoulder and a left shoulder positioned on a top portion of the cable support tray and wherein the cable has an outer cable diameter and wherein a distance between the right shoulder and the left shoulder is smaller than the outer cable diameter configured to provide an interference fit between the right shoulder and the left shoulder and the cable.

30. The PDT instrument of claim 23 further comprises a pair of threaded holes positioned in the front portion of the neck portion and wherein the at least one faster comprises a pair of threaded thumb screws rotatably captured in the front portion of the lid and wherein the pair of threaded thumb screws cooperate with the pair of threaded holes to releasably lock the lid in the closed position.

31. The PDT instrument of claim 26 wherein the plurality of photodetectors are each configured to produce an electrical signal based on a detected light transmitted from a respective one of the plurality of optical fibers.

32. The PDT instrument of claim 31 wherein the plurality of optical fibers are configured to be optically coupled to a conformable light applicator.

33. The PDT instrument of claim 32 wherein the plurality of therapy light sources are configured to be energized when the lid position switch is activated and when the cable position switch is activated.

34. The PDT instrument of claim 31 further comprising indicia positioned proximate the plurality of optical fiber connectors and the plurality of photodetectors in the base and matching indicia positioned on each of the plurality of optical fibers and the plurality of detector optical fibers.

35. The PDT instrument of claim 22 wherein the plurality of optical fibers and the plurality of detector optical fibers include an excess fiber length between an exit end of the cable and the plurality of optical fiber connectors and the plurality of photodetectors and wherein the at least one cable clamp and the cable support tray releasably capture the cable and provide strain relief to the plurality of optical fibers and the plurality of detector optical fibers in the closed position.

36. The PDT instrument of claim 22 wherein the hinge comprises:
a pair of base leaves positioned on a top portion of the base, the pair of base leaves including a respective tang;
a pair of lid leaves positioned on a top portion of the lid, the pair of lid leaves including a respective detent; and
wherein the respective tangs are configured to cooperate with the respective detent to releasable lock the lid in the open position.

37. The PDT instrument of claim 23 wherein the base, the lid and the cable cooperate to form a secure light enclosure when the at least one fastener releasably locks the lid in the closed position.

38. The PDT instrument of claim 21 further comprising a photodetector clamp to securely position the plurality of optical fiber connectors in the plurality of fiber connector apertures and the plurality of photodetectors in the plurality of photodetector apertures.

39. The PDT instrument of claim 26 further comprising a key switch mounted in the front portion configured to power on the PDT instrument.

40. The PDT instrument of claim 39 further comprising a laser power indicator light positioned on the front portion of the housing and configured to illuminate when the key switch is in an on positioned, the lid position switch is activated and the cable position switch is activated.

41. The PDT instrument of claim 40 further comprising an emergency stop button positioned on the front portion of the housing and configured to shut down the PDT instrument when activated.

42. The PDT instrument of claim 21 further comprising:
a microprocessor positioned within the housing configured to execute a treatment plan; and
a monitor screen positioned on the front portion of the housing.

43. The PDT instrument of claim 42 wherein the monitor screen comprises a graphical user interface configured to enable a user to control a plurality of parameters related to the treatment plan and at least one function of the plurality of therapy light sources.

44. The PDT instrument of claim 43 further comprising a data port positioned on the housing configured to provide communication between the microprocessor and an external device.

45. The PDT instrument of claim 44 wherein the external device is a data storage device configured to store the treatment plan.

46. The PDT instrument of claim 44 wherein the PDT instrument is configured to deliver the treatment plan to a patient via a conformable light applicator.

47. A method of delivering PDT to a patient comprising:
providing a PDT instrument comprising:
a housing having a front panel;
a plurality of therapy light sources positioned within the housing;
a multi-port connector panel positioned on a front portion of the housing comprising:
a base having opposing side walls connected to a top wall and a bottom wall and a back wall and a neck portion projecting from the bottom wall to define an interior;
a lid pivotably mounted to a portion of the top wall to selectively close the interior;
a plurality of optical fiber connectors positioned in a plurality of fiber connector apertures formed in the back wall;
a cable support tray positioned in a front portion of the neck portion; and
at least one cable clamp positioned in a front portion of the lid;
selectively coupling each of the plurality of optical fiber connectors to a respective one of the plurality of therapy light source;
selectively coupling each of the plurality of optical fiber connectors to a respective one of a plurality of optical fibers.

48. The method of delivering PDT to a patient of claim 47, wherein the PDT instrument further comprises a plurality of photodetectors positioned in a plurality of photodetector apertures formed in the back wall and wherein the method further comprises selectively coupling each of the plurality of photodetectors to a respective one of a plurality of detector optical fibers positioned in a cable.

49. The method of delivering PDT to a patient of claim 48, the PDT instrument further comprising a lid position switch mounted to the neck portion and a cable position switch positioned within the cable support tray, the method further comprising:
positioning the cable within the cable support tray and activating the cable position switch;
pivoting the lid into a closed position and activating the lid position switch;
clamping the cable between the at least one cable clamp and the cable support tray; and
releasably locking the lid in the closed position.

50. The method of delivering PDT to a patient of claim 49 further comprising optically coupling the cable to a conformable light applicator.

51. The method of delivering PDT to a patient of claim 50 wherein optically coupling the cable to a conformable light applicator comprises optically coupling each of the plurality of optical fibers to a respective one of a plurality of light emitters positioned in the conformable light applicator and optically coupling each of the plurality of detector optical fibers to a respective one of a plurality of fiber isotropic detectors positioned in the conformable light applicator.

52. The method of delivering PDT to a patient of claim 51 further comprising:
developing a treatment plan for a target tissue of the patient wherein the treatment plan includes a total dosage of a therapy light; and
inputting the treatment plan into the PDT instrument.

53. The method of delivering PDT to a patient of claim 52 further comprising positioning the conformable light applicator proximate the target tissue of the patient.

54. The method of delivering PDT to a patient of claim 53 further comprising:
powering on the PDT instrument;
delivering the therapy light to the target tissue of the patient using the plurality of light emitters;
monitoring the delivering of the therapy light to the target tissue of the patient using the plurality of fiber isotropic detectors to determine a dosage of delivered therapy light; and
comparing the dosage of delivered therapy light to the total dosage.

55. The method of delivering PDT to a patient of claim 54 further comprising completing the treatment plan when the dosage of delivered therapy light is substantially equal to the total dosage.

56. The method of delivering PDT to a patient of claim 49 further comprising providing strain relief to the plurality of optical fibers and the plurality of detector optical fibers in the closed position.

57. The method of delivering PDT to a patient of claim 49 further comprising forming a secure light enclosure in the closed position.

58. The method of delivering PDT to a patient of claim 54 further comprising illuminating a laser power indicator light when the PDT instrument is powered on, the lid position switch is activated and the cable position switch is activated.

59. The method of delivering PDT to a patient of claim 54 further comprising controlling the treatment plan using a graphical user interface.
